(19) United States
(12) Patent Application Publication
(10) Pub. No.: US 2024/0226594 A9
(43) Pub. Date: Jul. 11, 2024

(54) APPARATUS AND METHOD FOR NEUROMODULATION WITH MRI-GUIDED ULTRASOUND AND INDUCED ELECTRIC FIELDS

(71) Applicant: Weinberg Medical Physics, Inc., North Bethesda, MD (US)

(72) Inventor: Irving N. WEINBERG, Chevy Chase, MD (US)

(21) Appl. No.: 18/380,013

(22) Filed: Oct. 13, 2023

Related U.S. Application Data
(60) Provisional application No. 63/418,234, filed on Oct. 21, 2022.

(51) Int. Cl. A61N 2/06; A61N 2/00

(52) U.S. Cl. CPC A61N 2/06; A61N 2/006

(57) ABSTRACT

An apparatus and method may be provided utilizing electropermanent magnets (EPMs) to deliver either or both ultrasound and changing magnetic fields. The apparatus and method may be used to modulate neurons and/or transport materials in the nervous system of human or non-human animals.

### CROSS REFERENCE AND PRIORITY CLAIM

This application claims priority to U.S. Provisional Patent Application Ser. No. 63/418,234, entitled “APPARATUS AND METHOD FOR NEUROMODULATION WITH MRI-GUIDED ULTRASOUND AND INDUCED ELECTRIC FIELDS” filed Oct. 21, 2022, the entirety of which is incorporated by reference.

### FIELD

Disclosed embodiments are provided for diagnosing and treating diseases in animals, including humans. The disclosed embodiments may be used in medical and veterinary applications. The disclosed embodiments may be used outside of medicine as well, in the precise delivery of high-power electrical and ultrasound pulses.

### BACKGROUND

Both ultrasound and changing magnetic fields (which induce electric fields) have been used to modulate neuronal activity and transport therapeutic compounds (across the blood-brain barrier) in the brains of humans and non-human animals.

### SUMMARY

Disclosed embodiments describe the design and use of a system capable of delivering either or both ultrasound and induced electrical fields for neuromodulation and/or delivery of therapeutic compounds in the nervous systems of humans or non-human animals.

An apparatus and method may be provided utilizing electropermanent magnets (EPMs) to deliver either or both ultrasound and changing magnetic fields to modulate neurons and/or transport materials in the nervous system of human or non-human animals.

### BRIEF DESCRIPTION OF THE FIGURES

FIG. 1 shows an embodiment of an apparatus; and

FIG. 2 shows an embodiment of a method according to the disclosed embodiments.

### DETAILED DESCRIPTION

Disclosed embodiments relate to an apparatus and method utilizing an array of at least one EPM 10 and at least one diaphragm 40 to generate changes in a feature of an object under imaging guidance.

For the purposes of this specification, the term “feature” is intended to mean a structure of interest within an object of interest, for example where the feature is a tumor or section of brain within an object of interest such as a brain.

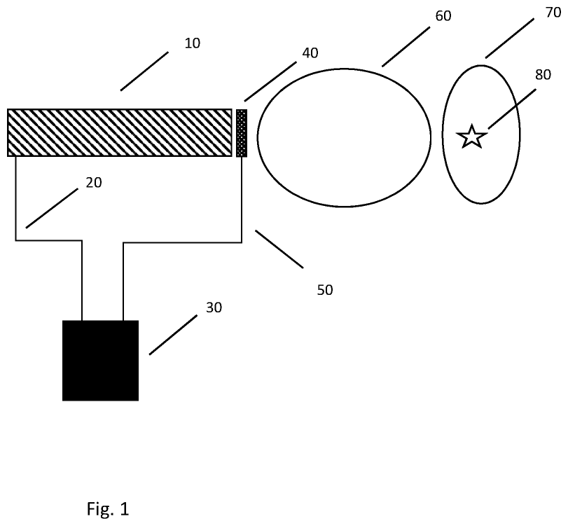

Referring to the figures, FIG. 1 shows an embodiment of the apparatus. At least one electropermanent magnet (EPM) 10 may be connected via cables 20 to a power supply and/or controller 30. It is understood that in general more than one electropermanent magnet are in the apparatus, but for clarity only one is shown in FIG. 1. At least one magnetizable diaphragm 40 may containing a coil or other material that can be magnetized and may be connected via cables 50 to a power supply and/or controller 30, which may be the same as the power supply for the electropermanent magnet. An interface 60, for example, a water bag, may be used to effectively couple sonic energy emitted by diaphragm 40 to an object 70, for example, a brain, with a target or feature of interest 80.

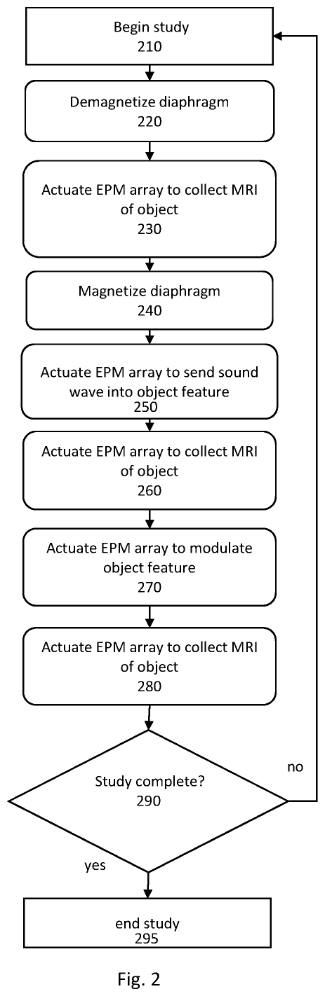

FIG. 2 shows an embodiment of a method of neuromodulation. In operation 210, a user begins a diagnostic study and/or therapeutic procedure of a human or non-human animal. In operation 210, diaphragm is demagnetized or otherwise prepared so as not to interfere with the EPM, while the EPM is used to collect an MRI of object 230, as taught, for example, by Weinberg U.S. Pat. No. 9,694,196, “SYSTEM, METHOD AND EQUIPMENT FOR IMPLEMENTING TEMPORARY DIAMAGNETIC PROPULSIVE FOCUSING EFFECT WITH TRANSIENT APPLIED MAGNETIC FIELD PULSES” incorporated by reference in its entirety. In operation 240, the diaphragm is magnetized or otherwise prepared to emit a sound wave when a magnetic field is applied. The EPM is used to generate a magnetic force on the diaphragm, thereby sending one or more sound wave into an object 250. The EPM may be used to generate an MRI of object 260, which may provide a measurement of the displacement of a feature in the object. The EPM may be used to modulate a neuron in an object 270. Alternatively, the EPM may be used to transport a magnetizable particle or have another physiological effect. The EPM may be used to collect an MRI of the object 280 subsequent the modulation, transport of a magnetizable particle or other EPM induced physiological effect. The user may decide to repeat one or more prior steps 290, or perform other steps, or that the study is complete 295.

It is understood that the timing of the one or more sonic pulses generated by an array of EPMs may be used to form a wavefront to implement neuromodulation for example by pressure waves, and that the pulses may be focused on a feature of interest through established phased ultrasound arrays methods.

The disclosed embodiments refer to prior publication by Weinberg 2022/0397625 A1 “APPARATUS AND METHOD FOR MAGNETIC RESONANCE IMAGING WITH ELECTROPERMANENT MAGNETS” incorporated by reference in its entirety.

It is understood that the diaphragm may have multiple sections in which each section may be differentially magnetized so that different sound wave phases and/or amplitudes may be generated by the forces applied to each section by the magnetic field produced by the EPM.

It is understood the diaphragm may include magnetic particles within a fluid, the particles being able to rotate under the influence of a magnetic field. The particles may be demagnetized through alternating magnetic fields, or may be polarized through application of a magnetic field by the EPM. A subsequent magnetic pulse that occurs in less than the rotation time of the magnetic particle will then have a propulsive force on the particles and on the net diaphragm. Other methods may be used to effect demagnetization or magnetization of the diaphragm.

For the purposes of this description, an electropermanent magnet is defined as a magnetizable material and a coil, whereby the magnetizable material retains some magnetization after an electrical current has passed through the coil.

It is understood that the EPM may be one of multiple EPMs (for example, in an array of EPMs), and may be combined with permanent magnets in the same or different arrays.

It is understood that the effect on tissues generated by the EPMs may be through induction of electric fields in the tissues, or through forces on magnetic particles, or through some other means.

An aspect of the disclosed embodiments is that the EPMs may be used to generate magnetic fields for the purpose of collecting images or neuromodulation, and also to generate sound waves for example, for the purpose of collecting images or neuromodulation or tissue ablation. An advantage of the disclosed embodiments is that having an MRI and ultrasound-generating system in the same device is convenient to users and that the near simultaneous application of magnetic and sonic fields may have synergistic effectiveness at neuromodulation.

Those skilled in the art will recognize, upon consideration of the above teachings, that the above exemplary embodiments and the controller may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out he above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

1. An apparatus comprising:
at least one electropermanent magnet and at least one magnetizable diaphragm, and
a controller that controls the electropermanent magnet to apply a magnetic field to an object of interest for the purpose of one of neuromodulation, imaging, or therapy, and also controls the electropermanent magnet to apply a magnetic force on the diaphragm that creates a sound wave that has an effect on a structure in the object of interest.

2. The apparatus as in claim 1 where the diaphragm contains more than one section, so that sound waves of different phases or amplitudes are created by the same diaphragm.

3. The apparatus of claim 1, further comprising an interface configured to couple the sound wave emitted from the diaphragm to the object of interest.

4. The apparatus of claim 1, wherein the at least one electropermanent magnet comprises an array of electropermanent magnets.

5. A method in which an electropermanent magnet is used to collect an image of an object of interest and then to generate a sound wave entering the object of interest, the method comprising:
controlling the electropermanent magnet to apply a magnetic field to the object of interest, and
controlling the electropermanent magnet to apply a force on a magnetizable diaphragm resulting in a sound wave.

6. The method as in claim 5 wherein the diaphragm contains more than one section, so that sound waves of different phases or amplitudes may be created by the same diaphragm.

7. The method of claim 5, further comprising an interface configured to couple the sound wave emitted from the diaphragm to the object of interest.

8. The method of claim 5, wherein the at least one electropermanent magnet comprises an array of electropermanent magnets.
